(19) United States
(12) Patent Application Publication
(10) Pub. No.: US 2023/0181369 A1
(43) Pub. Date: Jun. 15, 2023

(54) OPHTHALMIC SURGERY APPARATUS

(71) Applicants: UNIVERSITE DE BORDEAUX, BORDEAUX (FR); CENTRE NATIONAL DE LA RECHERCHE SCIENTIFIQUE - CNRS, PARIS (FR); CENTRE HOSPITALIER UNIVERSITAIRE DE BORDEAUX, TALENCE (FR); CENTRE TECHNOLOGIQUE ALPHANOV, TALENCE (FR)

(72) Inventors: John LOPEZ, GRADIGNAN (FR); Donetsi IRIBARREN, FLOIRAC (FR); Bruno CHASSAGNE, PESSAC (FR); David TOUBOUL, BORDEAUX (FR)

(21) Appl. No.: 17/995,560

(22) PCT Filed: Apr. 6, 2021
(86) PCT No.: PCT/FR2021/050599

§ 371 Date: Oct. 5, 2022

(30) Foreign Application Priority Data
Apr. 7, 2020 (FR) 2003451

(51) Int. Cl. A61F 9/008; A61B 90/00

(52) U.S. Cl. CPC A61F 9/0084; A61F 9/00827; A61F 9/00831; A61B 90/361; A61F 2009/0087; A61F 2009/00872; A61F 2009/00897; A61B 2017/0019

(57) ABSTRACT

The disclosure relates to an ophthalmic surgery apparatus for making an incision in ocular biological tissue such as a cornea or a crystalline lens. The apparatus includes: a laser source suitable for delivering a beam of laser pulses; an optical focusing system for focusing the beam of laser pulses on a focal point in the ocular biological tissue; an optical system for moving the beam of laser pulses, configured to move the focal point along a predetermined three-dimensional trajectory; a control unit configured to control the laser source, and the optical system for moving the beam of laser pulses, in such a way that the parameters of the beam of laser pulses and the parameters of the optical system for moving the beam of laser pulses are adjusted according to the position of the focal point in the trajectory during the incision.

### CROSS-REFERENCE TO RELATED APPLICATIONS

This application is a National Stage of International Application No. PCT/FR2021/050599, having International Filing Date of 6 Apr. 2021, which designated the United States of America, and which International Application was published under PCT Article 21(2) as WO Publication No. 2021/205111 A1, which claims priority from the benefit of French Patent Application No. 2003451, filed on 7 Apr. 2020, the disclosures of which are incorporated herein by reference in their entireties.

### BACKGROUND

### 1. Field

The disclosure relates to the field of eye-surgery apparatuses. More precisely, the disclosure relates to an ophthalmological surgical apparatus for assisting surgeons in performing corneal transplantations and refractive eye surgery.

The disclosure relates to an apparatus for performing eye surgery, by means of a picosecond laser.

In the present patent application, by picosecond laser, what is meant is a light source able to emit a laser beam composed of ultra-short pulses, the pulse duration of which is comprised between a few hundred femtoseconds and a few tens of picoseconds.

### 2. Brief Description of Related Developments

The cornea forms part of the outer shell of the eye. It is a transparent dielectric medium like glass, fashioned into a dome with almost parallel faces forming a biconvex aspherical dioptric interface. Its average diameter is about 12 mm and its average central thickness is about 540 microns. It has an average radius of curvature of about 7.8 mm. It allows incident light rays to be made to converge on the retina, with a view to forming an image thereon.

The cornea is made up of five distinct layers, from outermost to innermost:

the epithelium, which is composed of five to seven layers of constantly renewed stratified cells;
Bowman's membrane, which is acellular and non-renewable;
the stroma, which makes up 90% of the thickness of the cornea. It is composed of lamellae of collagen and of cells bathing in a slowly renewed gel matrix;
Descemet's membrane, which bears the endothelium;
the endothelium, which makes contact with the aqueous humor. The endothelium, which is the deepest layer, is made up of a unistratified layer of cells that do not renew.

As a result of accident or of a specific pathology, the cornea may become partially or totally opaque, thus degrading vision. Corneal transplantation is then the only effective way of restoring the function of the diseased cornea. It further allows good visual acuity to be restored and the pain caused by corneal lesions to be alleviated. Keratoplasty, or corneal transplantation, generally consists in extracting some or all of a pathological cornea and in replacing it with a healthy cornea from a donor. Currently, this operation is most often performed manually, using trephines and/or mechanical microkeratomes, by surgeons.

It is also possible to use refractive surgery to compensate for the group of visual disorders referred to as ametropia, these resulting in an inability to focus light rays onto the plane of the retina through the retina. Operable visual disorders of this type are myopia, hypermetropia, astigmatism and presbyopia. Lasers are currently used to help surgeons treat healthy corneas, to correct visual disorders.

Mention may be made here of the example of the LASIK technique, which consists in cutting, in the surface of the cornea, a flap of 90 to 120 microns thickness using a mechanical microkeratome (blade) or a femtosecond laser, then lifting this flap and reshaping the cornea below. ArF excimer lasers with ultraviolet radiation at 193 nm and a pulse duration of 10 to 25 ns allow very precise remodeling to be achieved (about 0.25 microns of photoablation per pulse). This procedure is currently the most practiced to treat myopia.

Mention will also be made of the particularity of the SMILE and RELEX techniques, which consist in cutting a lenticule of cornea from the thickness of the cornea, using a femtosecond laser, then in extracting it manually through a peripheral cut, thus avoiding the need to use an excimer laser. This procedure is fast-growing.

Currently, ophthalmological surgical apparatuses targeting operations in which the cornea is cut are mainly equipped with femtosecond lasers. A femtosecond laser is a laser which delivers pulses of duration comprised between 1 and a few hundred femtoseconds. The shortness of the pulses makes it possible to concentrate the laser energy in an extremely short time window, so as to achieve very high intensities, of the order of 10¹² at 10¹⁴ W/cm2, once focused on the target. The laser pulses are focused into the thickness of the cornea along a path that follows a straight or rectilinear line, so as to produce a cutting surface. The use of a femtosecond laser allows the energy deposited per pulse to be minimized, in order to avoid thermal effects while achieving an energy density sufficient to cause the appearance of a photodisruption-induced cavitation bubble.

A femtosecond laser generally requires complex laser circuitry and optical components to be used. In particular, in most available devices it is necessary to use a complex amplifying system that is very sensitive to its environment, in order to be able to amplify the laser radiation without damaging internal components. The cost of purchasing and maintaining femtosecond-laser-based ophthalmological surgical apparatuses remains relatively high.

The solution of transport through an optical fiber exists experimentally but remains very limited and constraining with respect to amplified femtosecond laser pulses.

Apparatus equipped with femtosecond lasers are generally very bulky and heavy. As a result, they are generally not very mobile and often require a dedicated room to be used, i.e. a room essentially devoted to refractive surgery, and therefore not usable for corneal transplantations or crystalline lens surgery.

Femtosecond lasers that do not use pulse amplification exist, but the low energy per pulse must then be compensated for by a focusing objective of high numerical aperture. This type of objective allows a micron-sized laser spot to be obtained in a very small working field, of the order of 1 mm in size. It is therefore necessary to use a motorized stage to move the laser beam in order to cover the area to be cut (which is close to 10 mm in diameter), this limiting the precision of the cuts despite the laser spot having a diameter of a few microns. For example, in the case of a LASIK operation, the cutting quality of the frontal plane (bed of the cut) is paramount. By quality, what is meant is the ease with which the cornea is cleaved and the roughness of the cutting planes. In the context of corneal incision, it is therefore essential to minimize this roughness by using the thinnest cutting planes possible. It is also important to guarantee the precision of the cut in order to limit diffraction and guarantee an optimal quality of vision. In this context, it will be noted that a laser spot with a diameter of 5 microns will cause photodisruption over 15 to 25 microns when an amplified femtosecond laser is used, and over less than 5 to 10 microns when an unamplified laser is used. As it would be desirable for the applied surgical actions to be of micron-order precision, the physics of the pulses and the optics of the system are crucial to achieving the expected surgical performance. Furthermore, the quality with which the edges are cut is important in corneal transplantation. However, with current ophthalmological surgical apparatuses, it is difficult to combine a high numerical aperture and a large working field and hence cover all the cutting paths under optimized conditions.

Moreover, current apparatuses for performing refractive surgical operations need an experienced surgeon if the operator and the machine are to correctly synchronize, such is the complexity and precision of the required actions. Practitioners thus require relatively lengthy training.

One objective of the present disclosure is therefore to provide an ophthalmological surgical apparatus that is particularly suitable for keratoplasty and ultra-high-resolution refractive corneal surgery. With respect to current devices, this apparatus is optimized in terms of ergonomics, compactness, robustness, lightness and mobility. Lastly, the efforts made to improve the automation of the tool will guarantee greater procedure safety, performance and versatility.

Another objective of the present disclosure is to provide an optical design and laser parameters specifically chosen to obtain an ophthalmological surgical apparatus able to produce tissue cuts that are as precise as possible, whatever the geometric constraints of the required paths.

### SUMMARY

In order to remedy the aforementioned drawbacks of the prior art, the present disclosure relates to an ophthalmological surgical apparatus for making a cut in an ocular biological tissue, such as a cornea or a crystalline lens, comprising:

a laser source suitable for delivering a pulsed laser beam,
a focusing optical system for focusing the pulsed laser beam to a focal point in the ocular biological tissue;
an optical system for moving the pulsed laser beam, said system being configured to move the focal point along a predetermined three-dimensional path;
a control unit configured to control the laser source and the optical system for moving the pulsed laser beam so that the parameters of the pulsed laser beam and the parameters of the optical system for moving the pulsed laser beam are adjusted depending on the position of the focal point on the path during cutting;
the parameters being the pulse duration of the laser beam, the energy per pulse, the pulse rate of the laser beam and the scan speed of the laser beam;
said control unit being configured to synchronously control the laser source and the moving optical system so that the pulse duration of the laser source varies according to the position of the focal point on the predetermined three-dimensional path.

Advantageously, said control unit is able to control the laser source so that the pulse duration of the laser beam is comprised between 350 femtoseconds and 3 picoseconds, and preferably comprised between 700 femtoseconds and 1.5 picoseconds.

According to another advantageous embodiment, said control unit is able to control the laser source so that the energy per pulse is comprised between 0.1 μJ and 20 μJ and the pulse rate comprised between 50 kHz and 2 MHz, and preferably between 50 kHz and 1 MHz.

Advantageously, said control unit is able to control the system for moving the pulsed laser beam so that the scan speed is comprised between 0.1 m/s and 10 m/s.

The features described in the following paragraphs may, optionally, be implemented. They may be implemented independently of one another or in combination with one another:

the system for moving the pulsed laser beam comprises a first scanner suitable for receiving the incident pulsed laser beam and configured to induce a movement of the pulsed laser beam along an axis Z, and a second scanner suitable for receiving the incident pulsed laser beam and configured to induce a movement of the beam in a plane (XY);
the focusing optical system is placed between the moving optical system and the biological tissue and configured to form, in the biological tissue, a focal point of diameter smaller than 8.5 μm, and preferably smaller than 6 μm, over a field of diameter comprised between 9 mm and 12 mm;
the focusing optical system consists of a telecentric optical combination having a numerical aperture comprised between 0.13 and 0.22, and preferably between 0.20 and 0.22;

According to one particularly advantageous embodiment, the ophthalmological surgical apparatus further comprises at least one camera configured to allow the cutting region to be viewed.

Preferably, said at least one camera is arranged between the focusing optical system and the biological tissue so that the incident imaging beam is inclined by an angle comprised between 30° and 50°, and preferably comprised between 45° and 47°, with respect to an optical axis of symmetry of the focusing optical system.

According to another particularly advantageous embodiment, the ophthalmological surgical apparatus further comprises a centering camera configured to center the optical axis of symmetry of the focusing system with respect to the biological tissue.

Preferably, the laser source used emits laser pulses at a wavelength comprised between 1020 nm and 1600 nm, and preferably between 1030 nm and 1090 nm.

According to one particular embodiment, the laser source being formed from two distinct parts, the first part comprising an oscillating laser cavity and a stretcher and the second part comprising an amplifying laser cavity, a compressor and an acousto-optical module, said apparatus comprises, on the one hand, a cutting module incorporating the focusing optical system, the optical system for moving the pulsed laser beam and the second part of the laser source, and on the other hand, a fiber-optic link for transmitting the laser beam generated by the first part of the laser source to the cutting module.

Advantageously, the ophthalmological surgical apparatus further comprises a flattening interface device comprising a plate with planar and parallel faces and/or a plano-concave plate.

The disclosure also provides an ophthalmological surgical equipment for making a cut in an ocular biological tissue, such as a cornea or a crystalline lens, comprising a self-balancing arm that is articulated about three axes X, Y and Z, and an ophthalmological surgical apparatus such as defined above, said arm having one end connected to a mobile electrotechnical rack and one end suitable for being coupled to the cutting module.

### BRIEF DESCRIPTION OF THE DRAWINGS

Other features, details and advantages of the disclosure will become apparent on reading the following detailed description, and on analyzing the appended drawings, in which:

FIG. 1 schematically shows a general view of an ophthalmological surgical apparatus according to one embodiment of the disclosure for cutting through a biological tissue, such as the cornea;

FIG. 2A schematically shows a front view of the arrangement of the two lateral viewing cameras;

FIG. 2B schematically shows a front view of the arrangement of the centering camera;

FIG. 3 shows an overview of an equipment of ophthalmological surgical equipment comprising a robot arm and a cutting module incorporating some of the elements of the ophthalmological surgical apparatus of FIG. 1;

FIG. 4 shows the formation of cavitation bubbles under the effect of impact of a laser beam in the cornea as a function of pulse duration for a path consisting of a spiral and of a helix;

FIG. 5 schematically shows three examples of paths associated with three types of cut;

FIG. 6 shows a series of images of side views of an irradiated region of a piece of glass, for pulse durations comprised between 330 fs and 3 ps, a wavelength set to 1030 nm, a pulse rate set to 500 kHz and an energy per pulse set to 2 μJ.

For the sake of clarity, similar elements have been designated by identical reference signs in all the figures.

In the context of the present patent application, by “picosecond laser”, what is meant is a laser that delivers pulses of duration comprised between a few hundred femtoseconds and a few tens of picoseconds.

In the context of the present patent application, by “energy density”, what is meant is an amount of energy per unit volume.

In the context of the present patent application, by “pulse rate”, what is meant is the number of pulses per second. Increasing the pulse rate allows treatment time to be decreased; however, undesirable effects may appear at high pulse rates. Specifically, when the time between two successive pulses is shorter than the thermal relaxation time of the target biological tissue, heat accumulates and the temperature of the tissue gradually increases. This thermal load induces a heat-affected region and/or a region of physico-chemical modification around the treated region. Therefore, an increase in pulse rate is necessarily accompanied by an increase in scan speed so as to keep spot formation contiguous in the focal plane and thus maintain the cutting quality.

Numerical aperture (NO)=n*sinθ, where n is the refractive index of the medium and θ the half-angle of incidence of the laser beam. A high numerical aperture allows a laser spot with a small diameter but with a shallower depth of field to be obtained. In contrast, a high numerical aperture generates a smaller working field. In the context of the present patent application, it is therefore essential to be able to combine a numerical aperture high enough to generate a spot of a diameter that is acceptable in the context of corneal cutting, and a sufficiently large working field, so as to be able to include the entire area of the pupil.

The threshold of optical breakdown or of photodisruption corresponds to the energy-per-pulse threshold from which cavitation bubbles form in a biological tissue. Photodisruption is a mechanism of conversion of the corneal tissue, when it receives ultra-short laser beam pulses. The corneal tissue in the region of impact is converted into a plasma that expands, forming a bubble cavitation. The expansion of this bubble leads to separation from the surrounding tissue. Generating thousands of bubbles in succession allows a (horizontal, vertical or oblique) cleavage plane to be produced at the desired depth in the cornea.

In the context of the present patent application, by “focal point”, what is meant is a region of impact of the laser beam corresponding to a place where the spot of the laser beam is formed in a focal plane.

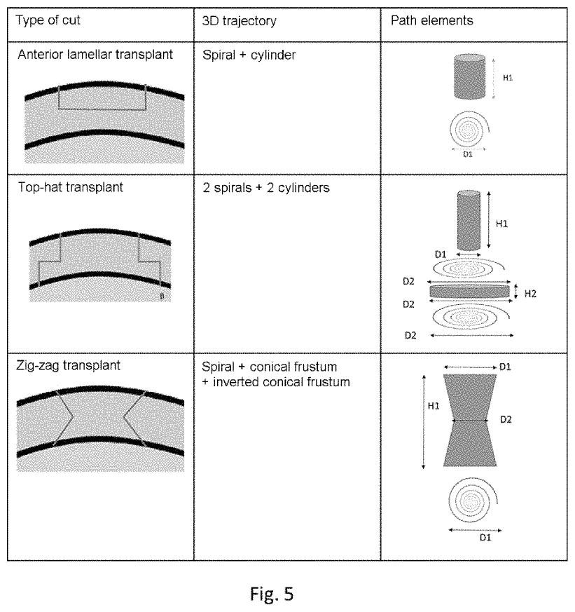

In the context of the present patent application, by “cutting surface”, what is meant is a surface comprising a set of contiguous points of impact forming a 2D or 3D geometric pattern. By way of example, in FIG. 5 the cutting surface of the first type of path is formed by a spiral and by a cylindrical surface.

In the context of the present patent application, by “cutting plane”, what is meant is a plane (XY) comprising a set of contiguous points of impact forming a 2D geometric pattern.

In the context of the present patent application, by “predetermined three-dimensional path”, what is meant is a set of points forming a curve generated by software. This curve is then decomposed into a set of contiguous vectors by the software of the system for moving the laser beam. The system for moving the laser beam moves the focal point of the laser beam along the predetermined path to form a series of points of impact in the cornea. The curve is described in the cornea starting from the deepest point in the cornea and ending at the point closest the front surface of the cornea. In other words, the starting point of the curve is located on the deepest cutting plane, and the end point of the curve is located on the cutting plane closest to the surface of the cornea. The curve describes cutting surfaces that when stacked form a cutting volume.

In the context of the present patent application, by “path element”, what is meant one portion of a 3D path. Each 3D path may be broken down into a plurality of path elements. By way of example, FIG. 5 shows three examples of 3D paths corresponding to three types of cut. In the case of a cylindrical anterior lamellar cut, the path may be formed from a spiral and a cylinder. The spiral corresponds to a path element here forming a cutting surface with a set of contiguous points forming a spiral pattern. The cylinder corresponds to another path element describing an ascending helix the pitch of which is adjustable. The cutting surface and the helix form a cutting volume. In the case of a top-hat cut, the path is formed from a spiral and cylinder, and from a spiral and cylinder. In the case of a zig-zag cut, the path is formed from a spiral and two conical frustums described by two facing helices of variable pitch and of variable radius. One set of the parameters consisting of pulse duration, energy per pulse, pulse rate and scan speed is associated with each path element.

### DETAILED DESCRIPTION

The drawings and the description below contain, for the most part, elements of a determinate nature. They will therefore not only serve to better understand the present disclosure but also contribute to the definition thereof, where appropriate.

Many LASIK apparatuses for performing surgery on the cornea are based on a femtosecond laser. Specifically, minimization of pulse duration is generally recommendable when cutting transparent biological tissues as it minimizes the volume in which energy is deposited and prevents heating of the corneal tissues liable to lead to irreversible damage thereto. Thus, to simplify the architecture of the laser source, and thereby increase its compactness and decrease its weight, it is difficult to envision the solution that consists of simply increasing the pulse duration. Specifically, the photodisruption threshold depends on intensity (W/cm²) or energy density (W/cm3). The longer the pulse duration, the higher the energy required to reach this threshold and the greater the risk of generation of thermal effects.

However, a key observation behind the present disclosure is that all ophthalmological surgical apparatuses using a femtosecond laser employ cutting parameters that remain the same throughout a given operation, i.e. everywhere on the 3D path of the beam and at every point in the cutting volume of the cornea. In particular, these prior-art apparatuses use a constant pulse duration during a given cutting operation. However, it has been found that, in the case of a femtosecond pulse, energy is generally deposited in a large volume upstream of the focal point and around the focal point. As a result, the energy density at the focal point is not necessarily optimal, i.e. maximum at the focal point.

In the present patent application, one of the objectives is to be able to deposit energy at the focal point, and therefore in a small volume, in order to maximize energy density. Optimizing energy density allows a cavitation bubble to be created with a minimum of energy. The optimization or maximization of energy density is achieved by dynamically varying pulse duration depending on the position of the focal point on the path 3 during the process of cutting the cornea.

The amount of energy deposited and the volume in which the energy is deposited vary as a function of pulse duration, and hence it is possible to act on the diameter of the cavitation bubbles with a view to guaranteeing the quality and precision of the cuts. Likewise, acting on bubble diameter has an effect on cutting speed, because the distance between two successive shots varies.

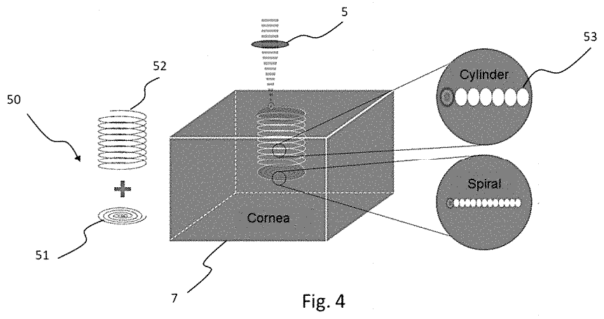

A pulse duration comprised between 1 and 2 ps promotes localized energy deposition, forming cavitation bubbles of small diameter. These small bubbles of a few microns allow a smoother cutting line to be formed, and thus the quality, precision and selectivity of the cut to be improved. In contrast, a pulse duration of a few hundred femtoseconds leads to a larger interaction volume, this improving cutting speed to the detriment of quality, as shown in FIG. 4. Specifically, a cutting surface formed from bubbles of larger diameter is rougher. A pulse duration comprised between 1 and 2 ps will therefore be used for path elements for which it is desired to promote cutting quality and to achieve a good selectivity in the direction of the axis Z. Conversely, a pulse duration of a few hundred femtoseconds will be used for path elements for which cutting speed is to be given priority.

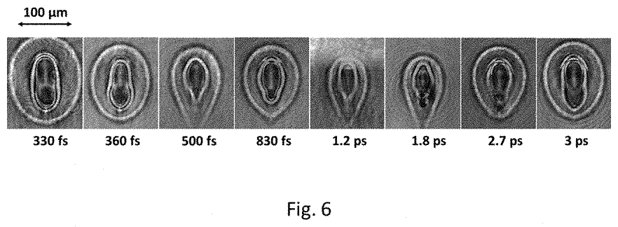

FIG. 6 shows a series of images that are side views of a region irradiated by depositing energy at a focal point. The irradiations were carried out with pulse duration varied between 330 fs and 3 ps and with the other parameters of the pulsed laser beam kept constant, i.e. wavelength at 1030 nm, pulse rate at 500 kHz, irradiation time at 500 ms, and energy per pulse at 2 μJ/s. The images show that volume decreases as pulse duration is varied from 330 fs to 1.8 ps, and, starting from 1.8 ps, surprisingly, an inversion in the variation in volume is observed as pulse duration is varied from 1.8 ps to 3 ps. The results of these irradiations here show that a pulse duration comprised between 1.2 ps and 1.8 ps allows cavitation bubbles of smaller volume to be formed.

In addition to dynamically varying pulse duration, pulse energy may also be adjusted on each path element so as to place it at the photodisruption threshold;

which depends on the depth of the focal point and on the transparency of the cornea.

Likewise, the pulse rate of the laser and the scan speed may also be adjusted so as to form contiguous photodisruption-induced cavitation bubbles.

The present disclosure therefore provides a surgical apparatus that is dedicated in particular to cutting the cornea, and that is based on the use of a laser the duration of the pulses of which is configurable on each path element depending on the desired cutting speed and quality.

The apparatus of the present disclosure is based on the use of a control unit that controls the laser source and the optical system for moving the laser beam, in order to associate an optimal pulse duration with each element of the 3D path of the focal point through the cornea, so as to promote either cutting quality via confinement of the deposited energy or cutting speed to the detriment of cutting quality. The control unit also allows an optimal energy, pulse rate and speed to be associated with each path element.

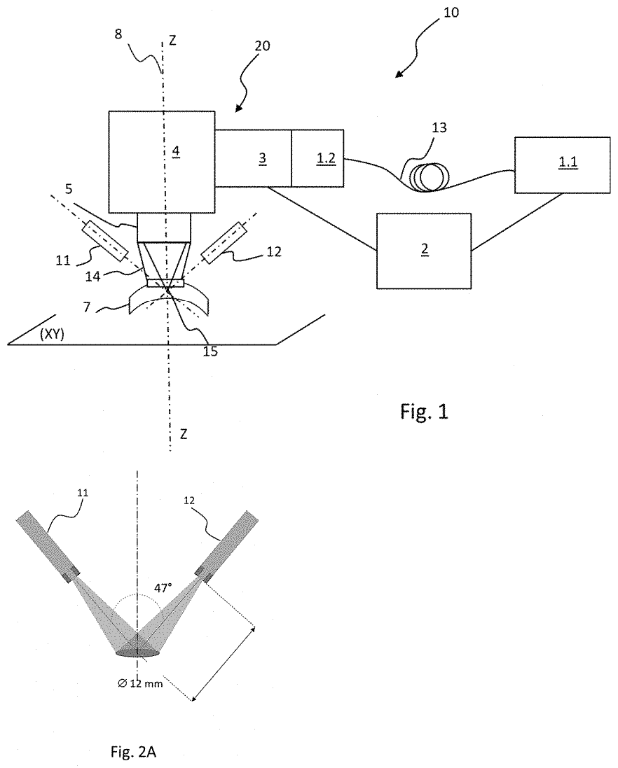

FIG. 1 schematically shows an ophthalmological surgical apparatus 10 according to one embodiment. The apparatus is positioned facing an eye to perform a surgical operation in which the cornea will be cut. A cross-sectional view of a cornea 7 having an external face and an internal face has been shown schematically here. An optical axis of symmetry 8 passing through the center of the cornea and perpendicular to the surface of the cornea is defined. The optical axis of symmetry 8 extends along a direction Z-Z orthogonal to a plane (XY). The cornea lies substantially in the plane (XY). The incident laser beam is focused at various depths in the volume of the cornea, in a direction parallel to the axis Z and with a normal angle of incidence.

The ophthalmological surgical apparatus 10 comprises a picosecond laser source suitable for delivering a pulsed laser beam, a focusing optical system 5 placed on the optical path of the pulsed laser beam and suitable for focusing the pulsed beam to a focal point 15 in the thickness of the cornea 7 and an optical system 3, 4 for moving the pulsed laser beam so as to move the focal point along a predetermined 3D path.

The laser source is formed from two distinct parts, a first part of the laser source 1.1 being composed of an oscillating laser cavity and of a stretcher, and a second part of the laser source 1.2 forming an amplifying laser head, the whole generating ultra-short pulses of the order of a few picoseconds to a few hundred femtoseconds. By way of example, to make a cut in a corneal tissue, the laser source may generate pulses of wavelengths comprised between 1030 nm and 1090 nm, of energy comprised between 0.5 μJ and 20 μJ, with a pulse rate comprised between 1 Hz and 2 MHz, and with a pulse duration comprised between 350 fs and 3 ps. A flexible optical fiber 13 conveys the non-amplified laser beam generated by the first part of the laser source 1.1 to the amplifying laser head 1.2, which comprises an amplifier, a compressor and an acousto-optical module, which have not been illustrated in FIG. 1.

In a known manner and in the context of the present disclosure, it is possible to act on the pulse duration output by the laser source by acting on the compressor so as to increase the distance between the two diffractive gratings of the compressor via a motorized translation stage. This action is controlled by a control unit 2.

As indicated above, when the laser beam impacts the medium of the cornea, a plasma is generated by ionization if the intensity of the laser is above a threshold value, called the threshold of optical breakdown or of photodisruption. A cavitation bubble then forms, causing very localized disruption of the surrounding tissues. Thus, a lamellar cut is made into the corneal tissue by producing a succession of small adjacent cavitation bubbles, which have a dimension larger than the diameter of the point of impact. These bubbles then form a cutting line. The path of the focal point of the laser beam is for example located on the surface of a cylinder or on a helicoid having an axial symmetry, and for example of elliptical or circular cross section and of determined dimension or diameter. The axis of the cylinder lies parallel to the axis Z-Z and is centered on the optical axis of symmetry of the cornea 8. The axis of the cylinder may also not be centered on the axis 8.

The optical system for moving the laser beam is used to move the focal point of the laser beam through the cornea along a predetermined path in the three directions X, Y and Z. It comprises a first scanner 3 allowing the focal point to be moved along the axis Z and a second optical scanner 4 allowing the focal point to be moved along the two axes X and Y in a focal plane (X, Y) corresponding to the cutting plane. The two scanners are coordinated and synchronized.

The first scanner 3 comprises an entrance pupil for receiving the laser beam delivered by the amplifying laser head 1.2, and an exit pupil, with a diameter for example of 25.4 mm, for sending the laser beam to the second scanner, and the scan speeds along the axis Z are comprised between 10 mm/s and 400 mm/s. By way of example, the first scanner is a motorized telescope that acts on the collimation of the laser beam and therefore on the position along the axis Z-Z of the focal point. The first scanner is for example one of the Varioscan scanners marketed by the company SCANLAB or the LS-scan Z scanner marketed by the company LASEA.

The second scanner 4, allowing the optical deflection of the beam, comprises an entrance pupil for receiving the laser beam from the first scanner and an exit pupil, with a diameter of 20 mm, for sending the laser beam to the focusing optical system 5. It for example comprises two pivoting optical mirrors mounted on pivoting axles, controlled by induction motors, allowing the laser beam to be deflected. The angular speed of each mirror corresponds to a linear speed on the target. The scan speeds obtained on the target, along the axis X and the axis Y, are comprised between 1 mm/s and 5000 mm/s.

The ophthalmological surgical apparatus comprises a control unit 2 that controls, in a synchronized manner, the laser source and the optical system for moving the laser beam, in order to dynamically vary a set of cutting parameters associated with each pulse, i.e. with each focal point or point of impact in the cornea along the predetermined 3D path, with a view to producing a cavitation bubble with a controlled size.

The control unit 2 allows pulse duration, scan speed, pulse rate, and energy per pulse to be varied dynamically depending on the position of the focal point or point of impact on the 3D path, in order to optimize both cutting quality and cutting speed.

The control unit is connected to the laser source and to the optical system for moving the laser beam by communication buses that make it possible to transmit, to the various elements of the apparatus, control instructions such as:

the activation signal of the laser source;
the pulse duration of the laser beam;
the energy per pulse;
the pulse rate;
the scan speed;
the 3D path.

According to one embodiment of the disclosure, the control unit 2 is configured to synchronously control the first scanner, the second scanner and the laser source via a software interface, in order to make the pulse duration of the laser source vary depending on the position of the focal point on the predetermined 3D path. The control unit 2 is configured to make, for example, the pulse duration vary between 350 fs and 3 ps, and preferably between 700 fs and 1.5 ps.

Likewise, the control unit may be configured to make the energy per pulse vary between 0.5 μJ and 20 μJ for each path element so as to place the focal point at the threshold of photodisruption corresponding to the threshold of optical breakdown. Specifically, the threshold of photodisruption is dependent on the depth of impact in the cornea and on the transparency of the cornea.

Likewise, the control unit may be configured to make the Z-speed vary, between 10 mm/s and 400 mm/s, and the XY-speed vary, between 1 mm/s and 5000 m/s, and the scan rate vary so as to juxtapose the bubbles induced by the successive focal points.

Prior to the cutting procedure, a predetermined three-dimensional path is loaded into the control unit. The 3D path is composed of a set of vectors extracted from a curve. These vectors correspond to the successive movements of the focal point through the cornea. Each laser pulse produces contiguous cavitation bubbles along these vectors. The distance between successive bubbles depends on the speed on the target and on the pulse rate of the laser. Preferably, speed relative to pulse rate is adjusted to obtain juxtaposed bubbles.

Advantageously, each path may be broken down into a plurality of path elements. It is therefore possible to associate one set of parameters such as pulse duration, energy per pulse, pulse rate and scan speed with each path element.

FIG. 4 schematically illustrates one example of a 3D path 50 formed from a first path element taking the form of a spiral 51, which is a 2D pattern, and of a second path element taking the form of a helix 52. Such a path is particularly suitable for making an anterior lamellar cut in the cornea.

The control unit controls the elements of the apparatus in order to associate a pulse duration of, for example, 1.5 picoseconds with all the laser impacts delivered on the first path element, to promote cutting quality, and to associate a pulse duration of, for example, 500 fs with all the laser impacts delivered on the second path element, to promote cutting speed.

The control unit controls the laser source and the optical system for moving the beam so as to form a first plurality of cavitation bubbles 53 the arrangement of which forms a spiral. Once this first plurality of cavitation bubbles has been produced, the control unit controls the laser source and the optical system for moving the beam so as to form a second plurality of cavitation bubbles 53 the arrangement of which forms a helicoidal pattern. As the example in FIG. 4 illustrates, the first bubbles formed have a smaller size than the second bubbles formed.

The focusing optical system 5 is configured so as to focus the laser beam in the thickness of the cornea such as to obtain points of impact of constant size (on the scale of a few microns) in a working field with a diameter of at least 10 mm. The optical axis of symmetry of the focusing optical system is centered on the optical axis of symmetry 8 of the cornea. According to another configuration, the optical axis of symmetry of the focusing optical system is not centered on the optical axis of symmetry of the cornea.

The focusing system 5 consists of an assembly of lenses. It will be noted that the optical designs here are not definitive, but are given, by way of indication, as an example of implementation fully meeting the specifications. Thus the number of lenses, their characteristics and their positioning could be different, while achieving the technical features defined above.

According to one embodiment of the disclosure, the focusing system is a telecentric optical combination. By way of example, the telecentric optical combination used may have the following characteristics:

wavelength: 1030 nm;
numerical aperture: 0.15;
working distance or focal length: 50 mm;
entrance pupil: 15 mm;
field size: 10 mm;
diameter of the focal point: 8.5 μm.

According to another advantageous embodiment of the disclosure, the focusing system is a telecentric optical combination having the following technical characteristics:

wavelength: 1030 nm;
numerical aperture: 0.22;
working distance or focal length: 30 mm;
entrance pupil: 14 mm;
field size: 10 mm;
diameter of the focal point: smaller than 6 μm, over the entire working field of 10 mm, i.e. at every point in the cornea.

This second telecentric optical combination makes it possible to employ a higher numerical aperture and therefore a focal point of smaller diameter, smaller than 6 μm; it therefore allows better precision and better cutting quality than the first lens, with which the diameter of the laser spot is about 8.5 μm.

Advantageously, the telecentric optical combinations of the present disclosure are configured so as to combine a high numerical aperture, of about 0.22, in order to obtain a precise and thin cut, and a large working field, of about 10 mm, in order to cover the entire surface of the functional pupil of the eye.

In addition, the use of this specific combination combined with an optimized pulse duration allows small bubbles of a few microns in a large working field of about 10 mm to be created, thus also improving the cutting quality (cutting surfaces of lower roughness).

Advantageously, the apparatus comprises a flattening interface device 14 placed in contact with the eye to be treated, which allows the angle of incidence of the laser beam on the cornea 7 to be decreased. This interface device for example comprises a plano-concave lens, the face placed facing the cornea of which has a radius of curvature larger than or equal to the average radius of curvature of the cornea. According to another embodiment, the interface device comprises a plano-planar lens. The optical axis of the flattening interface device is centered on the optical axis of symmetry 8 of the cornea. The interface device may be attached to the focusing optical system of the apparatus. In this case, the height of the flattening interface device is precisely equal to the focal length of the focusing optical system.

Advantageously and with reference to FIGS. 2A and 2B, the surgical apparatus comprises cameras 11, 12 allowing the surgeon to view the cornea on a display screen 40 during the operation, and a centering camera 9 the cone of vision of which is centered with respect to the optical axis of the cornea so as to correctly position the apparatus above the patient's eye.

As illustrated in FIG. 2A, the two viewing cameras 11, 12, which are equipped with a ring light, and positioned on either side of the focusing system 5, allow the eye to be illuminated and viewed during the operation. The diameter of the image region, which is about 12 mm, is larger than the cutting region through which the laser spot may be moved by the moving optical system. The two cameras allow the cutting region to be observed and the cutting process to be monitored. The two cameras are positioned between the focusing optical system and the cornea so that the imaging beam is oriented at an angle comprised between 45° and 47° relative to an optical axis of symmetry of the focusing system. These values allow the eye to be viewed in a field of 10.5 mm, while allowing for the bulk of the optics and mechanisms of the device, of the cameras and of the flattening interface device.

As illustrated in FIG. 2B, the centering camera 9 is a removable camera that is arranged between the focusing system and the tissue. It generates a cone of vision that is steered through 90° by a mirror 9.1 so as to generate a cone of vision 9.2 centered on the optical axis 8 of the focusing system, thus making it possible to position the apparatus above the patient's eye. Advantageously, the camera is equipped with a ring light that generates an annular alignment spot, thus allowing the patient to fixate on a spot of light centered on the optical axis of symmetry of the focusing system.

The three cameras thus allow the eye to be viewed before the operation, and monitoring to be carried out during the operation.

Advantageously, the ophthalmological surgical apparatus allows quality cuts to be made in the cornea with a pulsed picosecond laser. Such a laser is compatible with transmission via an optical fiber, unlike pulsed femtosecond lasers, which deliver pulses of intensity likely to damage the optical fiber. There are semi-rigid optical fibers that are able to carry these amplified laser pulses, but only up to a certain limit of energy per pulse. Furthermore, their optical transmission is not optimal, this inducing a significant loss in the power output from the optical fiber. Likewise, these optical fibers slightly alter the initial polarization of the laser beam. According to one advantageous embodiment of the present disclosure, the laser source is formed from two distinct parts 1.1 and 1.2. The first part 1.1 comprises an oscillating laser cavity and a temporal stretcher, and the second part, which is called the amplifying laser head 1.2, comprises an amplifying cavity, a compressor and an acousto-optical module, which have not been illustrated in the figures. A polarization-maintaining optical fiber 13 is interposed between the two distinct parts to transmit the beam. With such an architecture, the laser pulse output by the time stretcher is not amplified before it passes through the optical fiber. By virtue of this specific architecture, the optical system for moving the laser beam, the focusing optical system and the laser head are integrated into a cutting module 20, which is suitable for mounting at the end of a self-balancing articulated arm with a reach of 1 meter, thus freeing up space around the patient and limiting the floor space occupied by the apparatus.

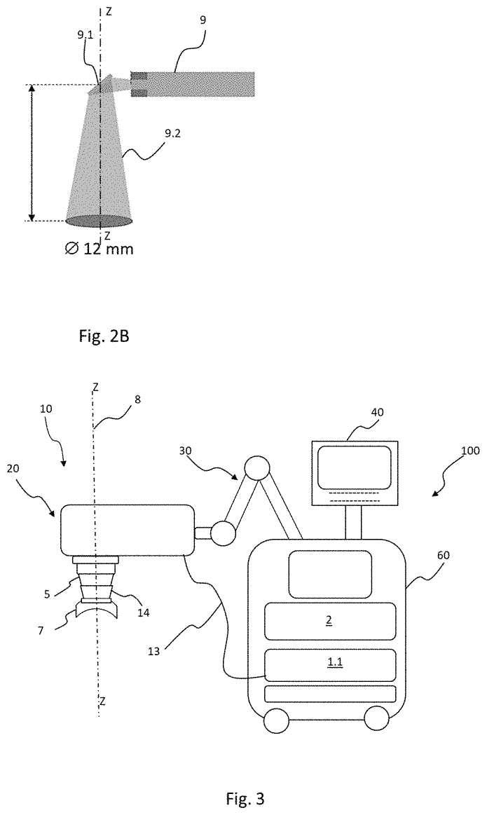

With reference to FIG. 3, the present disclosure also relates to an ophthalmological surgical equipment 100 in which the cutting module 20 is configured to be mounted at one end of a self-balancing articulated arm 30 with pneumatic assisting and blocking means, the other end of the arm being mounted on a mobile electrotechnical rack 60. Some of the elements of the equipment, for example the first part of the laser source 1.1, which comprises the oscillating laser cavity and the stretcher, the display screen 40 and the control unit 2 are housed in the mobile electrotechnical rack 60. The arm allows loads from 0 to 35 kg to be moved with substantially zero felt weight. The arm used is for example the Series 3 arm marketed by the company 3ARM. Actuators positioned on the cutting module allow the pneumatically blocked arm to be unlocked in order to move it in the three directions X, Y and Z. The self-balancing articulated arm allows the cutting module 20 to be positioned above the patient's eye. The positioning of the cutting module 20 is adjusted by the surgeon with the help of a centering camera 9.

The present disclosure allows an effective and compact ophthalmological surgical apparatus for making high-quality cuts in an ocular biological tissue, such as the cornea or the crystalline lens, to be provided. By virtue of a focusing system with a high numerical aperture, it is possible to produce spots of micron-order and uniform size over a field with a diameter of 10 mm and right through the thickness of the corneal layer. By adjusting the pulse duration of the laser beam depending on the position of the focal point on the path during cutting, it is also possible to adjust the size of the desired cavitation bubbles. The combination of spatial, temporal and dynamic shaping of the laser pulse allows quality and precision cuts to be obtained with a picosecond laser.

The ophthalmological surgical apparatus of the present disclosure has a relatively low manufacturing cost, since it is equipped with simple components that are less expensive than current apparatuses.

It is more compact and mobile by virtue of its specific architecture, in which the focusing system, the system for moving the beam and one part of the components of the laser source are integrated into a compact and light cutting module that may be mounted at the end of a self-balancing articulated arm.

A mobile electrotechnical rack may be used to house the rest of the equipment, such as one part of the laser source, and the display screen, with a view to easier movement of the whole assembly.

The use of a pre-established 3D path with optimized cutting parameters and a controlled system for moving the laser beam allows semi-automated cutting, ensuring reproducibility and repeatability.

One of the advantageous features of the technique provided in the present disclosure is use of a dynamic variation in cutting parameters, such as pulse duration, energy per pulse, the speed of movement of the beam and pulse rate, as a function of each path element, this allowing a precise and thin cut to be obtained, with cutting surfaces of low roughness, while guaranteeing the shortest possible cutting time. One technical advantage that results directly therefrom is the ability to cut thin layers and to thin a transplant with precision. Another technical advantage related to this continuous dynamic path, with low energy pulses, is better preservation of the corneal tissue by virtue of the very limited heat-affected regions generated thereby.

Another technical advantage is the ability to implement semi-automatic cutting throughout the operation. In contrast, centering of the apparatus, triggering of the laser beam and choice of the path are managed by the surgeon.

### INDUSTRIAL APPLICATION

The disclosure is particularly suitable for carrying out corneal cutting operations on donor and recipient with a view to a corneal transplantation. It may also be used to prepare and thin the transplant. It may be used for other LASIK, SMILE or RELEX operations without departing from the scope of the disclosure. For example, the disclosure is applicable to corneal refractive surgery such as the treatment of ametropia, in particular myopia, hypermetropia and astigmatism. The disclosure is also applicable to the treatment of cataracts with incision of the cornea. Generally, the disclosure relates to any operation on the cornea or, by extension of its capabilities, on the crystalline lens of a human or animal eye.

What is claimed is:

1. An ophthalmological surgical apparatus for making a cut in an ocular biological tissue, such as a cornea or a crystalline lens, comprising:
a laser source suitable for delivering a pulsed laser beam;
a focusing optical system for focusing the pulsed laser beam to a focal point in the ocular biological tissue;
an optical system for moving the pulsed laser beam, said system being configured to move the focal point along a predetermined three-dimensional path;
a control unit configured to control the laser source and the optical system for moving the pulsed laser beam so that the parameters of the pulsed laser beam and the parameters of the optical system for moving the pulsed laser beam are adjusted according to the position of the focal point on the path during cutting;
the parameters being the pulse duration of the laser beam, the energy per pulse, the pulse rate of the laser beam and the scan speed of the laser beam; and
said control unit being configured to synchronously control the laser source and the moving optical system so that the pulse duration of the laser source varies according to the position of the focal point on the predetermined three-dimensional path.

2. The apparatus as claimed in claim 1, wherein said control unit is able to control the laser source so that the pulse duration of the laser beam is comprised between 350 femtoseconds and 3 picoseconds, and preferably comprised between 700 femtoseconds and 1.5 picoseconds.

3. The apparatus as claimed in claim 1, wherein said control unit is able to control the laser source so that the energy per pulse is comprised between 0.1 μJ and 20 μJ and the pulse rate comprised between 50 kHz and 2 MHz, and preferably between 50 kHz and 1 MHz.

4. The apparatus as claimed in claim 1, wherein said control unit is able to control the system for moving the pulsed laser beam so that the scan speed is comprised between 0.1 m/s and 10 m/s.

5. The apparatus as claimed in claim 1, wherein the system for moving the pulsed laser beam comprises a first scanner suitable for receiving the incident pulsed laser beam and configured to induce a movement of the pulsed laser beam along an axis Z, and a second scanner suitable for receiving the incident pulsed laser beam and configured to induce a movement of the beam in a plane (XY).

6. The apparatus as claimed in claim 1, wherein the focusing optical system is placed between the moving optical system and the biological tissue and configured to form, in the biological tissue, a focal point of diameter smaller than 8.5 μm, and preferably smaller than 6 μm, over a field of diameter comprised between 9 mm and 12 mm.

7. The apparatus as claimed in claim 6, wherein the focusing optical system consists of a telecentric optical combination having a numerical aperture comprised between 0.13 and 0.22, and preferably between 0.20 and 0.22.

8. The apparatus as claimed in claim 1, which further comprises at least one camera configured to allow the cutting region to be viewed.

9. The apparatus as claimed in claim 8, wherein said at least one camera is arranged between the focusing optical system and the biological tissue so that the incident imaging beam is inclined by an angle comprised between 30° and 50°, and preferably comprised between 45° and 47°, with respect to an optical axis of symmetry of the focusing optical system.

10. The apparatus as claimed in claim 1, which further comprises a centering camera configured to center the optical axis of symmetry of the focusing system with respect to the biological tissue.

11. The apparatus as claimed in claim 1, wherein the laser source emits laser pulses at a wavelength comprised between 1020 nm and 1600 nm, and preferably between 1030 nm and 1090 nm.

12. The apparatus as claimed in claim 1, which further comprises a flattening interface device comprising a plate with planar and parallel faces and/or a plano-concave plate.

13. The apparatus as claimed in claim 1, wherein said laser source being formed from two distinct parts, the first part comprising an oscillating laser cavity and a stretcher and the second part comprising an amplifying laser cavity, a compressor and an acousto-optical module, said apparatus comprises, on the one hand, a cutting module incorporating the focusing optical system, the optical system for moving the pulsed laser beam and the second part of the laser source, and on the other hand, a fiber-optic link for transmitting the laser beam generated by the first part of the laser source to the cutting module.

14. An ophthalmological surgical equipment (100) for making a cut in an ocular biological tissue, such as a cornea or a crystalline lens, comprising a self-balancing arm that is articulated about three axes X, Y and Z, and an ophthalmological surgical apparatus as claimed in claim 13, said arm having one end connected to a mobile electrotechnical rack and one end suitable for being coupled to the cutting module.
